(19) United States
(12) Patent Application Publication
(10) Pub. No.: US 2026/0348936 A1
(43) Pub. Date: Nov. 5, 2026

(54) METHOD FOR QUANTIFYING NATURAL HYDROGEN GENERATION POTENTIAL FROM ROCK SAMPLES

(71) Applicant: Vinci Technologies, Nanterre (FR)

(72) Inventors: Jérémie Ammouial, Boulogne-Billancourt (FR); Nicolas Bouton, Villejuif (FR)

(21) Appl. No.: 19/491,019

(22) PCT Filed: Jun. 6, 2024
(86) PCT No.: PCT/EP2024/065644

§ 371 Date: Dec. 8, 2025

(30) Foreign Application Priority Data
Jun. 9, 2023 (FR) FR2305855

(51) Int. Cl. G01N 33/24

(52) U.S. Cl. CPC G01N 33/24

(57) ABSTRACT

The invention relates to a method for automatically quantifying dihydrogen generated by a sample of mineral or sedimentary parent rock containing a metal compound (such as iron), characterized in that a water reduction reaction is carried out in the presence of the sample and water vapor (2) at a temperature of approximately 1200 °C, and the amount of dihydrogen generated by the sample during this reaction is determined in order to qualify the parent rock as a potential source of natural hydrogen. The invention also relates to a device for carrying out said method.

The present invention relates to a method and a device for the continuous analysis of a hydrogen flow generated by chemical interaction between a water-laden carrier gas and a mineral rock sample.

Generally speaking, the object of the invention is to determine the amount of hydrogen generated by a rock sample during a water reduction reaction at high temperature and in the presence of this sample. The object of the invention is also to analyze the kinetics of the reduction reaction as a function of temperature and water quantity.

With declining hydrocarbon resources and growing global energy needs, and the quest for a less carbon-intensive energy mix, the worldwide production and use of hydrogen has become a major challenge.

In addition to being a raw material for the chemical industry, particularly for fertilizer production, hydrogen is also a fuel. The low greenhouse gas emissions produced by hydrogen combustion make it a good candidate as an environmentally-friendly resource in a context of global warming.

However, current hydrogen production remains highly dependent on fossil fuels. In fact, around 60% of the hydrogen produced comes from the vapor-reforming of methane (or natural gas) at high temperature, which in turn is most often produced using fossil fuels. About 20% comes from coal gasification and the rest from liquid HC. Less than 5% comes from electrolysis, a method that requires a great deal of electrical energy, which is usually produced by hydrocarbon or coal-fired power plants.

The main problem with hydrogen as a clean energy, or raw material, is therefore its production. Therefore, exploring sources of natural hydrogen makes perfect sense. Although still in the prospective stage, our understanding of natural dihydrogen generation systems should enable us to harness this natural hydrogen in the future without the need for fossil fuels. It is in this context that the invention provides solutions for simulating in the laboratory natural hydrogen production conditions in geological strata.

The applications targeted by the invention are mainly located in the field of exploration of natural hydrogen sources around the world. More specifically, the invention focuses mainly (but not exclusively) on a particular method for generating hydrogen from mineral rocks. This generation method results from the interaction between water, abundant in all deep geological environments, and the ferrous minerals present in certain types of rocks. This generation occurs naturally in various geological environments, such as in mid-ocean rifts, obduction zones, subduction zones and in some Archean and Neoproterozoic cratons. These environments are subjected to high temperatures ranging from 100 °C to 400 °C (and sometimes higher), thus promoting an oxidation-reduction reaction.

Hydrogen may be generated naturally by a plurality of chemical reactions. The best known is the serpentinization reaction, which characterizes the hydration of olivine to serpentine. This reaction generates hydrogen as a by-product. More precisely, water causes oxidation of the iron contained in fayalite (the ferruginous core of olivine representing around 10% of olivines) according to the following reaction:

2H₂O+3Fe₂SiO₄=2Fe₃O₄+3SiO₂+2H_{2 (g)}

A similar hydrogen-producing reaction also occurs during the hydration of orthopyroxene. In the cratons, it is the iron-rich rocks, particularly the BIFs (Banded Iron Formation), which will oxidize, the iron changing from ferrous (Fe²⁺) to ferric (Fe³⁺). The same reaction may take place in the presence of amphiboles, which are also iron-rich and found in granites.

As in the early days of oil exploration, the first signs of hydrogen generation underground were found on the surface. Indeed, some places in the world are known for their natural dihydrogen emanations, such as the Yanartas fires in Turkey, which have been burning for over 2,500 years. Other hydrogen emanations have also been spotted on the surface, associated with a singular geomorphology of circular depressions ranging in size from a few meters to several kilometers, known as “fairy circles”, and in which the vegetation is different.

In addition, an onshore natural hydrogen deposit was discovered by chance and has been exploited in Mali by Petroma (now Hydroma) since 2010. This shallow well feeds a turbine that generates electricity for a village of 1,500 inhabitants. Since then, numerous exploration permits have been applied for worldwide, including in the United States, France and Australia.

On the offshore side, certain geological environments have been identified as capable of generating natural hydrogen. This is true of all the mid-ocean ridges, but also of the Mariana Trench, where a plurality of serpentine mud volcanoes produce a gas mixing hydrogen and methane.

In this context, the invention consists of a method and a device capable of rapidly and automatically analyzing rock samples in the laboratory in order to determine the amount of hydrogen generated during a water reduction reaction in the presence of this sample. The invention thus makes it possible to deduce the remanent potential for natural hydrogen production from the rocks from which the sample was taken.

This object is achieved according to a first aspect of the invention by means of a method for automatically quantifying the dihydrogen generated by a sample of mineral parent rock containing a ferrous metal compound, characterized in that a water reduction reaction is carried out in the presence of the sample and water vapor from 300 °C to a temperature of approximately 1200 °C, and the amount of the dihydrogen generated by the sample during this reaction is determined in order to qualify the parent rock as a potential source of natural hydrogen.

One of the parameters sought by the method according to the invention is the temperature (TmH2) corresponding to the generation of a maximum amount of hydrogen and enabling the kinetics of said hydrogen generation to be determined.

Another parameter sought by the method of the invention is the molar yield (ηnH2) and/or mass yield (ηₘH2) of the water reduction reaction characterized by the ratio between the molar amount of hydrogen produced (nH2) and the molar amount of the iron-containing rock sample (n reagent) or its mass in the case of the mass yield, and which is defined according to the following formulas:

$${η}_{n} ( H 2 ) = \frac{n H 2 prod}{n reagent} \times 100 and {η}_{m} ( H 2 ) = \frac{m H 2prod}{m reagent} \times 100$$

The method of the invention thus uses the amount of hydrogen generated relative to the mass of sample used in the analysis to calculate useful molar and mass yields (ηₙH2 and ηₘH2) and to determine the hydrogen potential and maturity of the parent rock.

An advantageous feature of implementing this method is that the water is brought into contact with the rock sample by means of an inert carrier gas carrying water vapor.

The initial reactor temperature is between 200 °C and 400 °C and the final temperature of the reduction reaction is between 600 °C and 1200 °C.

Preferably, the temperature rise gradient is between 0.1 and 50 °C/min.

The carrier gas flow rate is between 0 and 500 mL/min, and its water content is monitored by raising the temperature of a liquid water reserve or by vaporizing liquid water in a hot gas flow.

At the reactor outlet, the gaseous effluents from the water reduction reaction are separated to ensure condensation of the water and drying of the gaseous effluent fraction.

This gaseous fraction is then analyzed to determine the amount of hydrogen produced by the sample.

The invention also relates to a device for implementing the method described above for the continuous quantification of hydrogen emitted by a sample of mineral or sedimentary parent rock, characterized in that it comprises a reactor intended to receive said sample, said reactor being provided with a carrier gas injection manifold fed by a tank containing water heated to between 0 and 100 °C and provided with a line for maintaining the temperature, heating means ensuring temperature rises from 0 to 1200 °C inside the reactor, a separator collecting the gaseous effluents from the reactor and ensuring condensation of the water and exhaust of the gaseous fraction of the effluents to a drying means and a hydrogen detector analyzing the dried gaseous fraction.

An advantageous feature of the invention is that the device comprises a piston for introducing the sample into the reactor.

In addition, the device comprises thermocouples intended for monitoring the temperatures of the tank and of the line for maintaining the temperature of the injected carrier gas, and a flowmeter for monitoring the flow rate of the injected carrier gas.

Preferably, the device of the invention comprises an injection nozzle controlling the amount of water injected into the reactor, and a hygrometer or dew point sensor intended for monitoring the water content of the injected carrier gas.

The invention makes it possible to provide accurate and reliable information to companies prospecting for hydrogen of natural origin from simple samples of parent rocks taken from the subsoil. In particular, this information provides a clear picture of a geological zone's potential for hydrogen production.

The quantification method may be implemented in the laboratory using a simple, easy-to-use device, including nomadic and/or delocalized operation.

The invention thus enables the hydrogen generation potential of a rock sample to be analyzed in less than an hour, whereas similar analyses in autoclaves, for example, may take weeks.

Other features and advantages of the invention will emerge on reading the following disclosure, with reference to the appended figures which are detailed hereinafter.

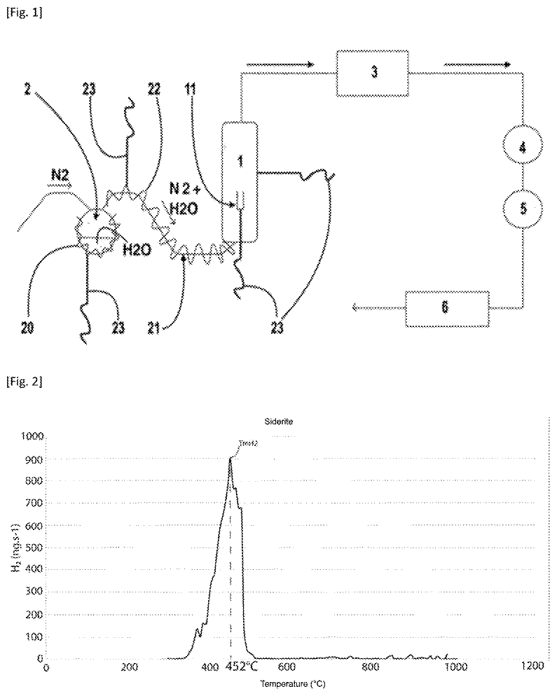

FIG. 1 is an embodiment of the device of the invention with the path of the carrier gas during implementation of the method.

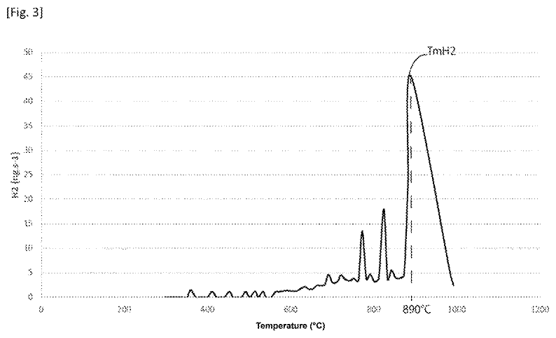

FIG. 2 is a graph showing hydrogen generation as a function of temperature from a siderite sample.

FIG. 3 is a graph showing hydrogen generation as a function of temperature from an olivine sample.

For greater clarity, identical or similar elements are identified by identical reference signs in the description and in the figures.

Naturally, the modes of implementing the method of the invention and of manufacturing the associated device shown schematically by the figures provided above and described hereinafter are given only by way of non-limiting example. It is explicitly provided within the framework of the invention that it is possible to propose and combine various modes to propose others.

Generally speaking, the invention relates to the field of prospecting for natural hydrogen sources by analyzing hydrogen parent rocks in the laboratory. More precisely, the method consists in automatically quantifying the dihydrogen generated by a mineral or sedimentary rock sample containing a ferrous metal compound during a water reduction reaction up to a temperature of 1200 °C. Determining the amount of dihydrogen generated by this sample thus enables the qualification of the parent rock as a potential source of natural hydrogen via the parameters ηH2 and TmH2.

To this end, water in vapor form is brought into contact with the rock sample in a reactor 1 by means of an inert carrier gas (e.g. nitrogen N₂), as illustrated in FIG. 1. Gaseous effluents from the high-temperature water reduction reaction are conveyed and separated to ensure condensation of the water and drying of the gaseous fraction. This dried gaseous fraction is then analyzed at the end of the method to determine the amount of hydrogen initially present in the sample.

The initial temperature of the water in vapor form injected into the reactor 1 is between 200 °C and 400 °C, and the final temperature of the reduction reaction is between 600 °C and 1200 °C.

Preferably, the temperature rise gradient in the reactor 1 is between 0.1 and 50 °C/min, so that hydrogen generation kinetics may be studied. The carrier gas flow rate injected into the reactor is between 0 and 500 mL/min, and the water content of the carrier gas is monitored by raising the temperature of a liquid water reserve.

Preferably, the temperature cycle integrated in the method starts at 300 °C and has a 3 min step. The temperature is then raised at a rate of 25 °C/min to a final temperature of 1200 °C.

An embodiment of the device used to implement this method is shown in FIG. 1. This device comprises a ceramic reactor 1 fed by a manifold 21 assuring the continuous injection of an inert carrier gas. This reactor is designed to withstand temperatures of up to 1200 °C, achieved by a coil of heating wires of which the windings have been judiciously distributed to obtain a homogeneous and precise temperature across the sample. The rock sample(s) is (are) introduced into the reactor 1 via an autosampler system, which deposits the sample(s) on a piston 11, ensuring that the sample(s) is (are) moved in translation through the reactor 1 and that it is sealed once closed.

The carrier gas is pre-loaded with water by means of a hydration/humidification system. To this end, the injection manifold 21 for the carrier gas is connected to a tank 2 containing water heated to between 0 and 100 °C. Thus, according to the embodiment of the method and by means of the device illustrated in FIG. 1, the carrier gas injected into the reactor 1 drives a quantity of water which is intended to react in the reactor 1 with the mineral phase of the rock sample.

The carrier gas hydration system comprises a tank 2 containing distilled water, located upstream of the pyrolysis reactor 1, in which the carrier gas bubbles. An optional gas humidity monitoring device may also be added to test different humidification rates. This tank 2 is equipped with thermal means 20 (such as joule-effect resistors) for heating the water from 0 to 100 °C to ensure its transition from the liquid phase to the vapor phase.

The manifold 21 running from the tank 2 to the inlet of the reactor 1 is itself maintained at a suitable temperature by means of a line 22 ensuring the temperature is maintained and thus avoiding any condensation prior to chemical interaction with the rock sample. The device of the invention also comprises a system of solenoid valves (not shown) of which the automatic activation enables the bypass of the humidification mode to be selected.

The carrier gas flow rate may be monitored using mass flowmeters (not shown) capable of varying flow rates from 0 to 500 mL/min.

In addition, the device comprises thermocouples 23 intended for monitoring the temperatures of the tank 2, the injected carrier gas manifold 21 and the reactor 1 (located below the sample and on the reactor wall), as well as a flowmeter (not shown) for monitoring the flow rate of the injected carrier gas.

Preferably, the device of the invention comprises an injection nozzle (not shown) controlling the parameters of the carrier gas as well as a hygrometer and/or a dew point detector (not shown) intended for monitoring the water content of the injected carrier gas.

At the outlet of the reactor 1, the device comprises a system 3 for separating the gaseous effluent coming from the pyrolyzed sample from the liquid fraction. These effluents are then passed through a drying medium such as a desiccant 4 to remove any residual water.

A hydrogen detector 6 performs the final analysis of the dried gaseous fraction, allowing the amount of hydrogen initially present in the sample to be determined. Depending on the type of hydrogen detector 6 used at the end of the line, a series of traps 5 may be added to remove elements likely to interfere with the measurement.

Examples of implementation of the method of the invention will now be described in detail hereinafter, applied to two different rock samples but under identical experimental conditions.

In these examples, the temperature of the tank 2 is maintained at 90 °C, as is the injection manifold 21. The carrier gas circulating in the water tank has a flow rate of 100 mL/min. The temperature cycle used starts at 300 °C with a 3 min temperature step and ends at 1000 °C. The temperature gradient used is 20 °C/min.

The two samples analyzed are, respectively, siderite (iron carbonate-FIG. 2) and olivine (main mineral component of the Earth's mantle-FIG. 3). The siderite, with the formula FeCO3, is of synthetic origin and 100% pure, with an iron content of around 48%. The olivine is of the natural forsterite type, with an iron content of around 3.8%.

When analyzing 120 mg of siderite, a significant hydrogen peak is generated between 350 °C and 500 °C, corresponding to the decarbonation range of this mineral (FIG. 2). The integration of this curve allows the production to be assessed at around 0.16 mg, or 0.08 moles of hydrogen per 1 mole of siderite analyzed, representing a yield of 8%. The hydrogen production then falls back to zero. In this example, the temperature (TmH2) is equal to 452 °C.

Analysis of 120 mg of forsterite produces less hydrogen, which is to be expected as this rock contains twelve times less iron than siderite. The molar yield this time is 0.6%. The hydrogen production starts at 600 °C, but peaks between 800 °C and 1000 °C (FIG. 3). In this example, the temperature (TmH2) is equal to 890 °C.

1. Method for automatically quantifying the dihydrogen generated by a sample of mineral or sedimentary parent rock containing an iron-containing metal compound, characterized in that a water reduction reaction is carried out in the presence of the sample and water vapor, the temperature of which has risen to approximately 1200 °C, and the amount of the dihydrogen generated by the sample during this reaction is determined in order to qualify the parent rock as a potential source of natural hydrogen.

2. Method according to claim 1, characterized in that the water is brought into contact with the rock sample by means of an inert carrier gas carrying water vapor.

3. Method according to claim 1, characterized in that the gaseous effluents from the water reduction reaction are separated to ensure condensation of the water and drying of the gaseous fraction of said effluents.

4. Method according to claim 3, characterized in that the dried gaseous fraction is analyzed to determine the amount of hydrogen initially present in the sample.

5. Method according to claim 1, characterized in that the initial temperature of the water vapor prior to the temperature rise is between 200 °C and 400 °C.

6. Method according to claim 1, characterized in that the final temperature of the water vapor at the end of the temperature rise during the reduction reaction is between 600 °C and 1200 °C.

7. Method according to claim 1, characterized in that the gradient of the temperature rise is between 0.1 and 50 °C/min.

8. Method according to claim 1, characterized in that the carrier gas flow rate is between 0 and 500 mL/min.

9. Method according to claim 1, characterized in that the water content of the carrier gas is monitored by raising the temperature of a water reserve or by vaporizing liquid water in a hot gas flow.

10. Method according to claim 1, characterized in that the temperature (TmH2) corresponding to the generation of a maximum amount of hydrogen is determined, and the kinetics of the generation of said hydrogen is deduced therefrom.

11. Method according to claim 1, characterized in that the quantity of hydrogen generated by the sample is used to calculate the useful molar and mass yields (ηₙH2 and ηₘH2) and to determine the potential amount of hydrogen in the parent rock, as well as the maturity of the parent rock from which said sample was taken.

12. Device for continuously quantifying the hydrogen emitted by a sample of mineral or sedimentary parent rock, characterized in that it comprises a reactor (1) intended to receive said sample, said reactor being provided with a carrier gas injection manifold (21) fed by a tank (2) containing water heated to between 0 and 100 °C and provided with a line (22) maintaining the temperature, a coiled reactor with heating wires ensuring temperature rises from 0 to 1200 °C inside said reactor, a separator (3) collecting the gaseous effluents from the reactor and ensuring the condensation of the water and exhaust of the gaseous fraction of the effluents to a drying means (4) and a hydrogen detector (6) analyzing the dried gaseous fraction.

13. Device according to claim 12, characterized in that it comprises a piston (11) ensuring the introduction of the sample into the reactor (1).

14. Device according to claim 12, characterized in that it comprises thermocouples (23) intended for monitoring the temperatures of the tank (2) and the line (22) for maintaining the temperature of the injected carrier gas.

15. Device according to claim 12, characterized in that it comprises a flowmeter for monitoring the flow rate of the injected carrier gas.

16. Device according to claim 12, characterized in that it comprises an injection nozzle controlling the amount of water injected into the reactor (1).

17. Device according to claim 12, characterized in that it comprises a hygrometer or dew point detector intended for monitoring the water content of the injected carrier gas.
